(19) United States
(12) Patent Application Publication
(10) Pub. No.: US 2006/0153908 A1
(43) Pub. Date: Jul. 13, 2006

(54) Spherical pellet formulations

(76) Inventors: Brian Strong, Merishausen (CH); Martin Kloemkes, Horgen (CH); Dieter Bachmann, Neuhansen (CH)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/519,018

(22) PCT Filed: Jun. 25, 2003
(86) PCT No.: PCT/EP03/06831

§ 371 Date: Jul. 15, 2005

(30) Foreign Application Priority Data
Jun. 27, 2002 (EP) 02077587.0

(51) Int. Cl. A61K 9/48; A61K 9/26; B27N 3/02

(52) U.S. Cl. 424/451; 424/469; 264/109

(57) ABSTRACT

A process for preparing spherical pellets comprising (a) a water-soluble active ingredient soluble, freely soluble or very soluble in water; and in particular having a water-solubility of ≧0.5 g/ml; (b) a spheronizing agent; (c) a dry lubricant, said method comprising preparing a mixture of the active ingredient, the spheronising agent, the dry lubricant; and an amount of water which is less than 5%, w/w relative to the total weight of the mixture; extruding said mixture to obtain an extrudate; and spheronising the extrudate to form spherical pellets. The invention further concerns pellets obtained by this process and sustained release oral dosage forms containing said pellets.

### FIELD OF THE INVENTION

This invention relates to a process for preparing spherical pellets containing a water-soluble drug, which pellets may be coated, and to pellets obtained by this process. It further concerns oral dosage forms containing said pellets.

### BACKGROUND OF THE INVENTION

Many pharmaceutical formulations come in single dosage unit forms, which allow the administration of discrete amounts of the active ingredient. The most frequently used unit dosage form without doubt is a tablet. In a number of instances there exists a need for higher or lower doses than the standard amount that is released upon administration of a single unit dose. In case of higher doses, several of the dose units can be administered or, if lower doses are required, the unit dosage form can be split, e.g. a tablet can be broken in half.

In a number of instances it may be required to administer the active ingredient in varying doses that do not fit into this pattern. This can for example be necessary for active ingredients that have to be administered in very specific quantities, e.g. quantities that are highly dependent upon the patient population at which they are aimed, or quantities that have to be adjusted in terms of weight, sex or age of the patient. In such instances it may be appropriate to use variable dosage forms or multi-unit dosage forms such as capsules or sachet. These dosage forms contain the required amounts of the active ingredient formulated in an appropriate carrier.

Obvious formulations for use in capsules or sachets are powdery formulations. However, it is not always possible or desirable to use powdery formulations for this purpose. The active ingredient may for example be too aggressive to the stomach or other parts of the gastro-intestinal system or may be prone to decomposition by gastric juices. In such instances the active ingredient needs to be kept separated from environmental factors by a suitable technique such as coating, e.g. by coating of granules, or by incorporation into pellets or beads. The latter in turn may also be coated for example, to provide further protection, for taste masking, or for affecting the release of the active ingredient.

Quite a number of active ingredients require specific release kinetics or prolonged release. In such instances use is made of so-called sustained or controlled release formulations.

Controlled release formulations have been introduced for active ingredients that require a specific release pattern such as a constant release during a certain period of time, i.e. a release of the active ingredient that is devoid of peaks or drops. A variety of controlled release formulations are now available that avoid temporary over- or under-dosing of the active ingredient.

Sustained release formulations have been developed in which the release of the active substance is prolonged in some way in order to maintain therapeutic activity for a longer period of time. Sustained release formulations typically are applied for drugs that have a short half-life or for active ingredients that require active blood plasma levels for long periods of time. In the former instance, multiple daily dose regimens can be avoided such as b.i.d., t.i.d. or q.i.d regimens, which often lead to problems caused by lack of patient compliance. Sustained release formulations are also applied for patients on chronic medication where one administration suffices to keep active blood plasma levels for longer periods such as several days or even weeks.

However, the term ‘sustained release’ is often also used for formulations that show controlled release during a prolonged period of time.

Also in the instance of sustained or controlled release formulations, the active ingredient can be incorporated into pellets, which may be coated with a suitable coating material that affects the release pattern of the active ingredient.

In order to have a regular and controllable release it is required that the pellets come in regular shapes, more in particular as regularly shaped spheres. An important factor that governs the release of an active from a pellet is the amount of the surface that is in contact with the medium to which the active ingredient is released. Irregularly shaped pellets have irregular surfaces, resulting in irregularities in the release of the active. Release of the active is better controllable with regularly shaped pellets.

A further advantage of spherically shaped pellets is that they are more easily coated and moreover that the thickness of the coating is more uniform when the pellets have regular round shapes. This is even more the case when the size distribution of the pellets is narrow. Still another advantage associated with spherically shaped pellets is their ease of handling and filling into capsules, sachets or other application forms such as bottles.

A further reason to prefer coated multi-unit dosage forms over coated single-unit dosage forms such as coated tablets, is the risk of dose dumping. This phenomenon occurs when there are undesired openings in the coating, which may be caused during manufacturing or by the patient while handling the dosage form, or by non-voluntary chewing on it. Small openings or cracks in the coating mantle causes contact of the interior with body fluids setting the release of the active in motion. The amount of active released in case of a single unit dose evidently will be much higher than with a multi-unit dosage form such as a pellet.

Spherically shaped pellets are usually produced by adding water to a dry blend of active ingredient and a suitable spheronizing agent and optional other ingredients and extruding the thus formed wet mass through a small orifice (typically approx. 1 mm). The water acts as a lubricant during this process and reduces the friction and heat generated during extrusion. Then the extruded material is placed into a spheronizer where it is spun at high speed. During this step the extrudates break and round into pellets, the size being determined by the size of the extrusion orifice. The extrudates need to be sufficiently moist to extrude, sufficiently dry to break and sufficiently moist to round without being too moist which results in congealing and sticking of the pellets. In this process step, the moisture content of the wetted mass is critical.

The amount of water required to enable extrusion of the dry blend typically is quite high. Upon spheronization a densification of the pellets occurs and the excess water in the extrudate migrates to the pellet surface where it causes sticking of the pellets to the spheronizer walls and plate.

When using this methodology, a number of active ingredients upon extrusion produce a sticky mass, which cannot be broken when spun at high speed. This seems to be the case when using water-soluble active ingredients. Apparently, such water-soluble active ingredients behave as an additional binder in the mixture and prevent the extrudate from becoming broken and rounded when put in a spheronizer. The extrudate in this instance rounds into inhomogeneously sized pellets.

One particular example of an active ingredient that is water-soluble and produces a sticky mass when subjected to a wet extrusion procedure is the analgesic tramadol, which typically is used in its hydrochloride salt form. Tramadol hydrochloride is very water-soluble and upon dissolution produces a sticky solution. This ingredient in particular behaves as an additional binder in the extrusion mixture and prevents the extrudate breaking when spun at high speed and rounding into inhomogenously sized pellets.

One of the problems associated with tramadol is that it has a relatively short half-life thus requiring a multiple dose regimen. Initial overdosing during the initial time period after administration may result in side effects whereas underdosing results in inefficacy so that the pain sensation may arise again. Overdosing problems may occur because tramadol hydrochloride is an orally active pure agonist opioid analgesic. Opioids have for many years been used as analgesics to treat severe pain. They, however, produce undesirable side effects and, as a result, cannot always be given repeatedly or at high doses. However, clinical experience indicates that tramadol lacks many of the typical side effects of opioid agonists, e.g., respiratory depression, constipation, tolerance and abuse liability. Tramadol's ‘atypical’ combination of non-opioid and opioid activity makes it a very unique drug.

Therefore, sustained release formulations of tramadol have been developed such as those described in EP-A-624366. However there is a need for sustained release formulations of tramadol of improved properties and spherical pellet formulations are an attractive option for such formulations. More in general there is a need for spherical pellet formulations of water-soluble active ingredients as well as convenient processes for preparing these.

Providing a process to produce spherical pellets containing water-soluble drugs that overcomes the above-mentioned difficulties and problems is an object of this invention.

### SUMMARY OF THE INVENTION

This invention relates to a process for manufacturing spherical pellets comprising

(a) a water-soluble active ingredient being soluble, freely soluble or very soluble in water;

(b) a spheronizing agent;

(c) a dry lubricant,

said method comprising preparing a mixture of the active ingredient, the spheronising agent, the dry lubricant; and an amount of water which is less than 5%, w/w relative to the total weight of the mixture; extruding said mixture to obtain an extrudate; and spheronising the extrudate to form spherical pellets.

Preferred is a process for manufacturing spherical pellets as specified herein wherein the active ingredient has a water-solubility of ≧0.5 g/ml.

In a preferred execution of the process the pellets are subsequently coated with a suitable coating.

In a further aspect the invention concerns spherical pellets obtainable or obtained by the process specified above or hereinafter.

In another aspect this invention concerns spherical pellets comprising

(a) a water-soluble active ingredient being soluble, freely soluble or very soluble in water;

having a water-solubility of ≧0.5 g/ml;

(b) a spheronizing agent;

(c) a dry lubricant.

Preferred are spherical pellets as specified herein wherein the active ingredient has a water-solubility of ≧0.5 g/ml.

Specific embodiments are spherical pellets in accordance with the present invention that are coated.

The pellets of the invention may be for application in immediate release products or, in particular, for sustained release products.

In another aspect, this invention relates to spherical pellets comprising a water-soluble active ingredient, said pellets having a low water content.

In still a further aspect, the invention concerns a pharmaceutical dosage form containing spherical pellets or a coated spherical pellets as described herein. A preferred such dosage form is a capsule filled with said pellets or coated pellets.

In still another aspect, the present invention concerns a sustained release pharmaceutical oral dosage form containing an effective amount of a water-soluble active ingredient, wherein the active ingredient is formulated into a spherical pellet as described herein.

In a preferred aspect of this invention, the active ingredient in the pellets is tramadol, or a pharmaceutically acceptable acid addition salt thereof.

In a more preferred embodiment, the invention relates to a sustained release oral pharmaceutical dosage form containing an effective amount of tramadol, or a pharmaceutically acceptable salt thereof, formulated into a spherical pellet, which has been coated with an appropriate sustained release coating. Of particular interest are capsules containing pellets as defined herein.

The sustained release oral dosage forms of this invention are for administering to a human patient on a twice-a-day (b.i.d.) and in particular on a once-a-day basis.

In still another aspect, the invention concerns a process for manufacturing a pharmaceutical dosage from, said method comprising filling the pellets into a suitable container. In a preferred aspect the container is a capsule.

Furthermore, the invention concerns a method of treating a warm blooded animal suffering from analgesia, said method comprising the administration of an oral dosage form containing an effective amount of tramadol, or a pharmaceutically acceptable salt thereof, said dosage form being as described herein.

### DETAILED DESCRIPTION OF THE INVENTION

Unless indicated otherwise, any percentage is weight by weight (w/w), relative to the total weight of the composition or to the total weight of the pellet. Whenever used, a singular includes a plural and vice versa a plural includes a singular.

The term ‘spherical pellet’ is meant to comprise pellets, beads or spheroids that are more or less of regular shape. In particular embodiments of the invention the shape is round or about round, i.e. having or approaching the shape of a small sphere.

The average size of the pellets may vary but preferably the diameter is in the range of about 0.1 mm to about 3 mm, in particular from about 0.5 mm to about 2 mm, more preferably about 1 mm.

The size distribution of the pellets may vary but in general it is preferred that it has limited variation. It may vary between within a range of 10 to 20%. The size distribution may vary in a statistical manner, i.e. in a bell-shaped curve wherein e.g. 90% or e.g. 95% of the number of pellets may be within a size range that varies between about 10% to about 20% of the average sizes mentioned above.

The active ingredients incorporated into the pellets of this invention are soluble, freely soluble or very soluble in water. The terms soluble, freely soluble or very soluble in particular are as defined in the European Pharmacopeia. The latter specifies ‘soluble’ as the water solubility of an active ingredient being in the range of from 10 to 30 ml per gram solute; the term ‘freely soluble’ the water solubility of an active ingredient being in the range of from 1 to 10 ml per gram solute; the term ‘very soluble’ the water solubility of an active ingredient being less than 1 per gram solute. Preferred active ingredients in the spherical pellets in accordance with the present invention as well as in the process for preparing said spherical pellets, the dosage forms according to the invention, the process for preparing said dosage forms, the methods of treatment or any other feature or aspect according to this invention, are those active ingredients that are freely soluble or very soluble. Of particular interest are those active ingredients having a water-solubility of ≧0.5 g/ml.

Particular active ingredients are those forming a sticky mass upon contact with water and/or the other excipients used in the extrudate mixture. More in particular, the active ingredients used in the pellets according to this invention are those that act as an additional binder in the mixture that ia extruded and spheronized. Examples of water-soluble active ingredients are tramadol hydrochloride, chlorpromazine hydrochloride, diphenhydramine hydrochloride, metamizole sodium, econazole nitrate, rabeprazole sodium, galantamine hydrobromide, terconazole nitrate, and the like.

The active ingredient is present in an amount, which is in the range of from about 0.1 to about 50%, in particular from about 1 to about 45%, more in particular from about 10 to about 40%, or from about 20% to about 40%, or from about 30% to about 35%, w/w relative to the total weight of the pellet.

The pellets of the invention further comprise a spheronising agent that may be any suitable pharmaceutically acceptable material, that is capable of forming, together with the active ingredient, spherical pellets. A preferred spheronising agent is microcrystalline cellulose. The microcrystalline cellulose used may suitably be, for example, the product sold under the tradename ‘Avicel™’. The spheronising agent is present in an amount, which is in the range of from about 15% to about 90%, in particular from about 20% to about 70% w/w, more in particular from about 30% to about 50%, or from about 35% to about 45%, relative to the total weight of the pellet.

Optionally the pellets may contain other pharmaceutically acceptable carriers or ingredients such as binders, bulking agents and colorants. Suitable binders, some of which may also contribute to the controlled release properties of the pellets, include water-soluble polymers, e.g. water-soluble hydroxyalkyl celluloses such as hydroxypropyl cellulose, or water insoluble polymers, such as acrylic polymers or copolymers, or alkyl celluloses such as, for example, ethyl cellulose. Suitable bulking agents include lactose or colloidal silicon dioxide. The amount of these other ingredients in the pellets will be relatively small, e.g. lower than about 30%, or about 20%, or lower than about 10% or about 5% w/w relative to the total weight of the pellet.

The pellets also contain a dry lubricant. Apart from providing lubrication, the dry lubricant also allows the material to be extruded at a much lower moisture content thereby reducing the sticking observed in the spheronizer.

The dry lubricant in particular is a mono-, di- or triglyceride, or any mixtures thereof. Suitable mono-, di- or triglycerides are the mono-, di- or triesters of glycerine and one or more fatty acids. The mono-, di- or triglycerides may contain the same or different fatty acid residues or mixtures thereof, e.g. technical mixtures obtained from saponification of natural oils. Of particular interest are fatty acid triglycerides wherein the fatty acid residue has from 12 to 30 carbon atoms and is saturated or partially unsaturated, and wherein the fatty acid residue may be substituted, e.g. with one or more hydroxy functions. Preferred are mono-, di- or triglycerides derived from C₁₈₋₃₀ fatty acids, in particular derived from C₂₂₋₂₆ fatty acids. Of particularly preferred interest are behenic acid mono-, di- or triglycerides. Particularly suitable mono-, di- or triglyceride mixtures are those wherein the diglyceride component is predominantly present, e.g. at >50% w/w, or even at >70% of the glyceride mixture.

The dry lubricant preferably is solid at room temperature and has a melting point or melting range which is in the range of about 60° C. to about 90° C., in particular is in the range of about 70° C. to 80° C.

A particularly suitable dry lubricant is the glyceride mixture sold under the trade name ‘Compritol™ 888ATO’ which is a mixture of glyceryl mono-, di- and tribehenate, the dibehenate fraction being predominant, and having a melting range of about 69-74° C.

Preferably, the dry lubricant is selected such that it does not impact the dissolution behavior of the active ingredient.

The dry lubricant is present in an amount, which is in the range of from about 2% to about 50%, in particular between about 10% and about 40%, more in particular between about 20% and about 40% w/w, or from about 30% to about 35% relative to the total weight of the pellet.

The pellets according to the invention have a low water content. In particular embodiments, the water contents in the pellets is lower than 5%, more in particular lower than 3%, still more in particular lower than 2% w/w relative to the total weight of the pellet.

Particular embodiments of the present invention are spherical pellets comprising

(a) from about 0.1 to about 50% of a water-soluble active ingredient which is soluble, freely soluble or very soluble in water and preferably having a water-solubility of ≧0.5 g/ml;

(b) from about 15% to about 90% of a spheronizing agent;

(c) from about 2% to about 50% of a dry lubricant.

Further particular embodiments of the present invention are spherical pellets comprising

(a) from about 1 to about 40% of a water-soluble active ingredient which is soluble, freely soluble or very soluble in water and preferably having a water-solubility of ≧0.5 g/ml;

(b) from about 20% to about 70% of a spheronizing agent;

(c) from about 10% to about 35% of a dry lubricant.

Further particular embodiments of the present invention are spherical pellets comprising

(a) from about 1% to about 50% of tramadol or a pharmaceutically acceptable acid-addition salt thereof, in particular tramadol hydrochloride;

(b) from about 15% to about 90% of micro-crystalline cellulose;

(c) from about 10% to about 40% of a glyceryl fatty ester, in particular C₂₂₋₂₆ fatty acid mono-, di- or triglycerides.

More particular embodiments of the present invention are spherical pellets comprising

(a) from about 10% to about 40% of tramadol hydrochloride;

(b) from about 20% to about 70% of micro-crystalline cellulose;

(c) from about 20% to about 40% of a glyceryl fatty ester, in particular C₂₂₋₂₆ fatty acid mono-, di- or triglycerides.

Still more particular embodiments of the present invention are spherical pellets comprising

(a) from about 20% to about 40% of tramadol hydrochloride;

(b) from about 20% to about 40% of micro-crystalline cellulose;

(c) from about 20% to about 35% of a glyceryl fatty ester, in particular C₂₂₋₂₆ fatty acid mono-, di- or triglycerides;

(d) less than about 5% of water.

The pellets according to the invention may be made by an extrusion process followed by spheronization. Thus in a further aspect, the present invention provides a process to produce spherically shaped pellets containing water-soluble active ingredient as defined herein, said process comprising extrusion of a suitable mixture containing the active ingredient, the spheronizing agent and dry lubricant, followed by a spheronization step.

The mixture used in the extrusion process may, apart from the above-mentioned active ingredient, spheronizer and dry lubricant, also comprise one or more suitable carrier materials and other optional ingredients. The amounts of each of the ingredients in the extrusion mixture accords with the amounts in the end product as specified herein. A small amount of water may be added to the mixture. The water content preferably is kept at a low level, in particular the water content is reduced such that the extrusion mixture is dry or almost dry. Instead of water, the extrusion mixture contains a dry lubricant as outlined above, allowing the material to be extruded at a much lower moisture content thereby reducing the sticking observed in the spheronizer. In a particular execution, the amount of water is about 5% or lower, or about 3% or lower, or about 1.5% or lower, w/w, relative to the total weight of the mixture for extrusion.

The ingredients in the extrusion mixture may be mixed together in any given sequence. In one type of embodiments, the dry lubricant is added to a mixture of the active ingredient and the spheronizer material with a small amount of water, at room temperature. The mixture is subsequently extruded through a small orifice. The diameter of the latter is in relation to the size of the pellets that are eventually produced from the extrudate. In certain embodiments, the diameter of the orifices is in the range of about 0.5 mm to 2.0 mm. The extrusion may be done at slightly elevated temperature but preferably is performed without applied heating. The extrudated material is subsequently placed into a spheronizer where it is spun at high speed.

In specific embodiments of the invention, the pellets are subsequently coated with a suitable coating using art known methods. The coating can either be a functional coating or a diffusion controlling coating.

A functional coating may be applied for e.g. taste masking, protection of the pellets, to have improved stability (shelf-life) or for identification (for example by coloring). Functional coating often will be film coating, using any film coating material conventional in the pharmaceutical art. Preferably an aqueous film coating is used.

Diffusion controlling coatings are designed to achieve a target release profile such as controlled or sustained release. Suitable controlled or sustained release coating materials include water-insoluble waxes and polymers such as polymethacrylates, for example the Eudragit™ polymers, or water insoluble celluloses, in particular alkyl celluloses such as ethylcellulose. Optionally, water-soluble polymers such as polyvinylpyrrolidone or water-soluble celluloses such as hydroxypropylmethyl-cellulose or hydroxypropylcellulose may be included. Further components that may be added are water-soluble agents such as polysorbate. Of particular interest is ethylcellulose (‘EC’). Preferably, a suitable plasticizer is added. A coating material that is particularly suitable is the coating material sold under the trade name Surelease™ (Colorcon), which is a dispersion of ethylcellulose.

In particular embodiments, the active ingredient for use in the pellets according to the present invention is tramadol, which is the compound (1R,2R or 1S,2S)-2-[(dimethylamino)methyl]-1-(3-methoxyphenyl)-cyclohexanol which belongs to a class of analgesic cycloalkanol-substituted phenol esters having a basic amine group in the cycloalkylring, disclosed in U.S. Pat. No. 3,652,589. Preferably tramadol is used as a pharmaceutically acceptable salt form, in particular as its hydrochloride salt. Tramadol is commercially available from Gruenenthal or may be made by the process described in U.S. Pat. No. 3,652,589.

Because of the bitter taste of the tramadol active ingredient, the pellets may be coated for taste-masking purposes although this may be of less importance if the pellets are used in a capsule dosage form.

The pellets of the present invention can be administered as such, if desired as controlled or sustained release formulations, but most preferably are incorporated into suitable oral dosage forms. Therefore, in a further aspect, the invention provides unitary oral dosage forms, which comprise pellets as described herein in an amount that is such that the dosage form contains an effective amount of the active ingredient incorporated into the pellets. Examples of such dosage forms are sachets. A preferred dosage form is a capsule.

In still another aspect, the present invention provides a process for manufacturing an oral dosage from comprising spherical pellets as specified herein, said process comprising filling the spherical pellets into a suitable container, e.g. into a sachet or capsule.

In particular embodiments, the invention provides unitary dosage forms, which comprise tramadol hydrochloride pellets as described herein in an amount that is such that the dosage form contains an effective amount of tramadol hydrochloride. Particular embodiments of such dosage forms may contain from about 10 mg to about 100 mg tramadol hydrochloride per unit, preferably from about 15 mg to about 75 mg of tramadol hydrochloride per unit, or from about 25 mg to about 65 mg of tramadol hydrochloride per unit.

The spherical pellets of the invention and in particular the oral dosage forms in which they are incorporated, have a particular dissolution rate in vitro, said dosage forms providing an effective therapeutic effect for a sufficiently long period of time, in particular for at least 12 hours more in particular for about 24 hours after oral administration. This more specifically is the case with spherical pellets and dosage forms containing tramadol hydrochloride. The oral dosage forms of the invention and more in particular the dosage forms containing tramadol hydrochloride, may be suited for dosing every 12 or every 24 hours.

The present invention provides pellets that are of sufficient spherical homogeneity so that they can conveniently be coated resulting in coated pellets that can be used in sustained or controlled release applications. Additionally, the pellets of this invention have a narrow size distribution and are such that they may have a coating of homogeneous thickness.

The pellets of this invention and the dosage forms derived thereof are sustained release formulations that release the active ingredient, and in particular tramadol, in a controlled manner, i.e. without peaks or drops in its release pattern.

In a specific aspect, the present invention provides a method for treating conditions of pain, in particular severe pain, in mammals, said method comprising administering spherical pellets wherein the active ingredient is tramadol or a pharmaceutically acceptable salt thereof, in particular tramadol hydrochloride, said pellets being as specified herein, said pellets being administered in an amount effective to treat said conditions of pain or severe pain. Preferably, said method comprises administering the pellets in suitable oral dosage forms, e.g. in capsules, comprising an effective amount of spherical pellets in accordance with the present invention.

### EXAMPLES

### Example 1

A dry blend of 1400 g of tramadol hydrochloride, 1400 mg of microcrystalline cellulose and 1200 g of glyceryl benehate (Compritol 888 ATO™, Gattefosse) is wet massed with approximately 60 g of water and extruded through a small orifice (approx. 1 mm). The extruded material is placed into a spheronizer where it is spun at high speed (pellet speed of between 5 and 20 ms⁻¹). During this step the extrudate breaks and rounds into pellets, the size being determined by the size of the extrusion orifice. The extrudate breaks easily and produces round pellets of uniform size at a much reduced moisture level and no sticking is observed in the spheronizer. The pellets are coated uniformly with 120 g Opadry II™ (a dry blend of polymers and polysaccharides available from Colorcon) followed by 2400 g Surelease™.

The thus prepared spherical pellets are filled into capsules using standard filling equipment.

### Example 2

Dissolution Rate:

The in vitro dissolution rate of the preparation of example 1 was measured according to Ph. Eur. Paddle Method (USP App. 2) at 75 rpm. The dissolution tests were performed on the capsules in 900 ml 0.05 M phosphate buffer with a pH value of 6.8 (USP) at 37° C. A sinker device was used to avoid the floating of the capsules in the vessel. The detection was performed by using the high performance liquid chromatography (HPLC) with a refractive index detector for the detection of the active compound. For an in situ measurement of the release rate, a fiber optic dissolution system was used, using the second derivative correction method at the wavelength range of 283 to 289 nm. The dissolution profile can be described as follows:

About 10% Tramadol released after 1 hour,

About 25% Tramadol released after 2 hours,

About 45% Tramadol released after 4 hours,

About 67% Tramadol released after 8 hours,

About 80% Tramadol released after 12 hours,

About 90% Tramadol released after 18 hours

About 100% Tramadol released after 24 hours by weight.

1. A process for manufacturing spherical pellets comprising
(a) a water-soluble active ingredient which is soluble, freely soluble or very soluble in water;
(b) a spheronizing agent; and
(c) a dry lubricant;
said method comprising preparing a mixture of the active ingredient, the spheronising agent, the dry lubricant; and an amount of water which is less than 5%, w/w relative to the total weight of the mixture; extruding said mixture to obtain an extrudate; and
spheronising the extrudate to form spherical pellets.

2. A process according to claim 1 wherein the pellets are subsequently coated with a suitable coating.

3. A spherical pellet comprising
(a) a water-soluble active ingredient which is soluble, freely soluble or very soluble in water;
(b) a spheronizing agent; and
(c) a dry lubricant.

4. A spherical pellet according to claim 3 comprising
(a) from 0.1 to about 50% of a water-soluble active ingredient which is soluble, freely soluble or very soluble in water;
(b) from about 25% to about 90% of a spheronizing agent; and
(c) from about 2% to about 50% of a dry lubricant.

5. A spherical pellet according to claim 3 comprising
(a) from about 1% to about 40% of a water-soluble active ingredient which is soluble, freely soluble or very soluble in water;
(b) from about 20% to about 70% of a spheronizing agent; and
(c) from about 10% to about 35% of a dry lubricant.

6. A spherical pellet according to claim 3 comprising
(a) from about 1% to about 50% of tramadol or a pharmaceutically acceptable acid-addition salt thereof, in particular tramadol hydrochloride;
(b) from about 15% to about 90% of micro-crystalline cellulose; and
(c) from about 10% to about 35% of a glyceryl fatty ester, in particular a C22-26 fatty acid mono-, di- or triglycerides mixture.

7. A spherical pellet according to claim 3 wherein the pellet has a low water content.

8. A spherical pellet according to claim 3, wherein the active ingredient in the pellets is tramadol, or a pharmaceutically acceptable acid addition salt thereof.

9. A spherical pellet according to claim 3 that is coated.

10. A pharmaceutical dosage form containing spherical pellets or coated spherical pellets as claimed in claim 3.

11. A dosage form according to claim 10, which is a capsule filled with pellets or coated pellets as claimed in claim 3.

12. A process for manufacturing a pharmaceutical dosage form as claimed in claim 11, said method comprising filling the pellets into a suitable container, or in particular into a capsule.

13. The process according to claim 1 wherein the water-soluble active ingredient has a water-solubility of greater than 0.5 g/ml.

14. The process according to claim 3 wherein the water-soluble active ingredient has a water-solubility of greater than 0.5 g/ml.

15. The process according to claim 4 wherein the water-soluble active ingredient has a water-solubility of greater than 0.5 g/ml.

16. The process according to claim 5 wherein the water-soluble active ingredient has a water-solubility of greater than 0.5 g/ml.

17. A spherical pellet according to claim 6 wherein the pellet has a low water content.

18. A spherical pellet according to claim 6 wherein the active ingredient in the pellets is tramadol, or a pharmaceutically acceptable acid addition salt thereof.

19. A spherical pellet according to claim 6 that is coated.

20. A pharmaceutical dosage form containing spherical pellets or coated spherical pellets as claimed in claim 6.
